(19) United States
(12) Patent Application Publication
(10) Pub. No.: US 2005/0191360 A1
(43) Pub. Date: Sep. 1, 2005

(54) Particles for inhalation having rapid release properties

(75) Inventors: Richard P. Batycky, Newton, MA (US); Michael M. Lipp, Framingham, MA (US)

Correspondence Address: ELMORE CRAIG & VANSTONE, P.C., 209 MAIN STREET, N. CHELMSFORD, MA, 01863, US

(73) Assignee: Advanced Inhalation Research, Inc., Cambridge, MA (US)

(21) Appl. No.: 11/051,497

(22) Filed: Feb. 4, 2005

Related U.S. Application Data
(60) Provisional application No. 60/543,246, filed on Feb. 10, 2004.

(51) Int. Cl. A61K 38/29; A61K 9/14

(52) U.S. Cl. 424/489; 514/2

(57) ABSTRACT

The invention generally relates to formulations having particles comprising phospholipids, bioactive agent and excipients and the pulmonary delivery thereof. Dry powder inhaled PTH formulations are disclosed. Improved formulations comprising DPPC, PTH and sodium citrate which are useful in the treatment of lowered PTH conditions are disclosed. Also, the invention relates to a method of for the pulmonary delivery of a bioactive agent comprising administering to the respiratory tract of a patient in need of treatment an effective amount of particles comprising a bioactive agent or any combination thereof in association, wherein release of the agent from the administered particles occurs in a rapid fashion. Kits containing receptacles of the formulations are also disclosed.

### RELATED APPLICATION

This application claims the benefit of U.S. Provisional Application No. 60/543,246, 10 filed on Feb. 10, 2004. The entire teachings of the above application(s) are incorporated herein by reference.

### BACKGROUND OF THE INVENTION

Pulmonary delivery of bioactive agents, for example, therapeutic, diagnostic and prophylactic agents provides an attractive alternative to, for example, oral, transdermal and parenteral administration. That is, pulmonary administration can typically be completed without the need for medical intervention (self-administration), the pain often associated with injection therapy is avoided, and the amount of enzymatic and pH mediated degradation of the bioactive agent, frequently encountered with oral therapies, can be significantly reduced. In addition, the lungs provide a large mucosal surface for drug absorption and there is no first-pass liver effect of absorbed drugs. Further, it has been shown that high bioavailability of many molecules, for example, macromolecules, can be achieved via pulmonary delivery or inhalation. Typically, the deep lung, or alveoli, is the primary target of inhaled bioactive agents, particularly for agents requiring systemic delivery.

The release kinetics or release profile of a bioactive agent into the local and/or systemic circulation is a key consideration in most therapies, including those employing pulmonary delivery. That is, many illnesses or conditions may require administration of a consistent level or rapid delivery of a bioactive agent to provide an effective therapy. Typically, this can be accomplished through a multiple dosing regimen or by employing a system that quickly releases the medicament.

For better regulation of PTH levels in humans, a need exists for formulations suitable for efficient inhalation comprising bioactive agents, for example, PTH, and wherein the bioactive agent of the formulation is released in a manner that is at least as efficient as presently available treatments and prophylactics, especially for the treatment of conditions where a subject is in need of PTH, for example, one of the above listed conditions where lower than normal levels of PTH are found in a subject.

A need also exists for formulations suitable for delivery to the lung and rapid release into the systemic and/or local circulation. Such formulations are expected to increase the willingness of patients to comply with prescribed therapy, and may achieve improved disease treatment and control.

Importantly, there exists a need for formulation which can ensure fast release of the bioactive agent while maintaining chemical stability of the bioactive agent.

### SUMMARY OF THE INVENTION

Formulations of the invention ensure fast release of the bioactive agent while maintaining both chemical stability of the bioactive agent and physical stability of the particle formulations, the latter two of which are challenging in light of the hygroscopicity of parathyroid hormone. That is, the Applicants have achieved the difficult three-way combination of (a) fast release, (b) maintainence of parathyroid hormone chemical stability and (c) maintenance of particle formulation physical stability via protection of the hygroscopic bioactive agent. With respect to (c), maintenance of particle formulation physical stability is of particular importance in relation to consistency and reproducibility of pulmonary delivery of parathyroid hormone formulations. For example, in order to ensure fast release and reproducibility of systemic levels of parathyroid hormone achieved upon delivery, the formulations of the invention are designed to disperse consistently across a range of both inhalation flowrates and environmental conditions, including elevated humidities.

Formulations having particles comprising, by weight, approximately 60% to approximately 94% DPPC, approximately 1% to approximately 20% PTH and approximately 5% to approximately 20% sodium citrate are disclosed. In another embodiment, the present invention is a formulation having particles comprising, by weight, about 85% DPPC; about 5% PTH; and about 10% sodium citrate; a formulation having particles comprising, by weight, about 80% DPPC; about 10% PTH; and about 10% sodium citrate; or a formulation having particles comprising, by weight, about 70% DPPC; about 20% PTH; and about 10% sodium citrate.

In another embodiment, the invention is a formulation having particles comprising, by weight, from about 1% to about 89% DPPC; from about 1% to about 20% PTH; from about 5% to about 20% sodium citrate and from about 5% to about 90% leucine. In another embodiment, the particle comprise, by weight, about 16% DPPC; about 12.5% PTH; about 10% sodium citrate; and about 55.5% leucine; about 13% DPPC; about 6% PTH; about 10% sodium citrate; and about 71% leucine; or about 12% DPPC; about 16% PTH; about 10% sodium citrate; and about 62% leucine.

The present invention also features methods for treating a human patient in need of PTH comprising administering pulmonarily to the respiratory tract of a patient in need of treatment, an effective amount of particles comprising by weight, approximately 60% to approximately 94% DPPC, approximately 1% to approximately 20% PTH and approximately 5% to approximately 20% sodium citrate, more particularly, about 85% DPPC; about 5% PTH; and about 10% sodium citrate; or alternatively about 80% DPPC; about 10% PTH; and about 10% sodium citrate; or alternatively about 70% DPPC; about 20% PTH; and about 10% sodium citrate.

The present invention also features methods for treating a human patient in need 25 of PTH comprising administering pulmonarily to the respiratory tract of a patient in need of treatment, an effective amount of particles comprising by weight, from 1% to about 89% DPPC; from about 1% to about 20% PTH; from about 5% to about 20% sodium citrate and from about 5% to about 90% leucine. In another embodiment, the particle comprise, by weight, about 16% DPPC; about 12.5% PTH; about 10% sodium citrate and about 55.5% leucine; or about 13% DPPC; about 6% PTH; about 10% sodium citrate and about 71% leucine; or about 12% DPPC; about 16% PTH; about 10% sodium citrate; and about 62% leucine.

This method is particularly useful for the treatment of conditions characterized by lower than normal levels of PTH [lower PTH conditions] such as but not limited to (1) autoimmune destruction of the parathyroid gland, (2) hypomagnesemia; (3) hypoparathyroidism; (4) metastatic bone tumor; (5) milk-alkali syndrome (excessive calcium ingestion); (6) sarcoidosis; and (7) vitamin D intoxication. If desired, the particles can be delivered in a single, breath actuated step.

The present invention also features methods for treating a human patient in need of PTH comprising administering pulmonarily to the respiratory tract of a patient in need of treatment, an effective amount of particles comprising by weight, approximately 60% to approximately 94% DPPC, approximately 1% to approximately 20% PTH and approximately 5% to approximately 20% sodium citrate, more particularly, about 85% DPPC; about 5% PTH; and about 10% sodium citrate; or alternatively about 80% DPPC; about 10% PTH; and about 10% sodium citrate; or alternatively about 70% DPPC; about 20% PTH; and about 10% sodium citrate or an effective amount of particles comprising by weight, from 1% to about 89% DPPC; from about 1% to about 20% PTH; from about 5% to about 20% sodium citrate and from about 5% to about 90% leucine. In another embodiment, the particle comprise, by weight, about 16% DPPC; about 12.5% PTH; about 10% sodium citrate and about 55.5% leucine; or alternatively about 13% DPPC; about 6% PTH; about 10% sodium citrate and about 71% leucine; or alternatively about 12% DPPC; about 16% PTH; about 10% sodium citrate; and about 62% leucine; wherein release of the PTH is rapid. This method is particularly useful for the treatment of lowered PTH conditions. If desired, the particles can be delivered in a single, breath actuated step.

In addition, the present invention features methods of delivering an effective amount of PTH as described above and administering via simultaneous dispersion and inhalation the particles, from a receptacle-having the mass of the particles, to a human subject's respiratory tract, wherein release of the PTH is rapid. Particularly useful for rapid release are formulations comprising low transition temperature phospholipids.

The present invention also features methods of delivering an effective amount of PTH to the pulmonary system, comprising providing a mass of particles comprising by weight, of from about 1.5 mg to about 20 mg, preferably in a receptacle. The mass of particles comprise a mass of PTH of about 50 micrograms to about 2 mg per receptacle, more particularly, about 100 micrograms to about 1 mg of PTH per receptacle. The particles are administered from a receptacle having the mass of the particles, to a human subject's respiratory tract, wherein release of the PTH is rapid. Particularly useful for rapid release are formulations comprising low transition temperature phospholipids.

The invention also features a kit comprising two or more receptacles comprising unit dosages selected from the PTH formulations described herein, either alone or in combination with other bioactive agents, in particular, antiresorptive agents. Combinations of receptacles containing different formulations within the same kit are also a feature of the present invention. For example, the kit can comprise two or more receptacles comprising unit dosages of particles comprising by weight, from about 60 to about 94% DPPC; from about 1 to about 20% PTH; and from about 5 to about 20% sodium citrate more particularly, about 85% DPPC; about 5% PTH; and about 10% sodium citrate; or alternatively about 80% DPPC, about 10% PTH; and about 10% sodium citrate; or about 70% DPPC; about 20% PTH; and about 10% sodium citrate. Alternatively, the kit can comprise two or more receptacles comprise unit dosages of particles comprising, by weight,, from about 1 to about 89% DPPC; from about 1 to about 20% PTH; about 5 to about 20% sodium citrate; and from about 5 to about 90% leucine, more particularly, about 16% DPPC; about 12.5% PTH; about 10% sodium citrate; and about 55.5% leucine; or about 13% DPPC; about 6% PTH; about 10% sodium citrate; and about 71% leucine; or about 12% DPPC; about 16% PTH; about 10% sodium citrate; and about 62% leucine. Receptable of both kits can be combined with one another.

The present invention also features a kit comprising at least two receptacles each receptacle containing a different amount of dry powder PTH suitable for inhalation.

In another aspect, the invention features a formulation having particles comprising, by weight 75% DPPC; 15% PTH; and 10% sodium citrate, wherein the method of preparing the formulation comprises preparing a solution of DPPC; preparing a solution of PTH and sodium citrate; heating each of the solutions to a temperature of 50° C.; combining the two solutions such that the total solute concentration is greater than 3 grams per liter (e.g., 5, 10, or 15 grams/liter); and spray drying the combined solution to form particles. In one embodiment, the solute concentration of the combined solution is 15 grams per liter.

In another embodiment, the above-described particles comprise a mass of from about 1.5 mg to about 20 mg of PTH containing particles (for example, 1.0, 1.5, 2.5, 5, 7.5, 10, 12.5, 15, 17.5, 20, or 25 mg). In one embodiment, for a therapeutically relevant dose of PTH in the blood of the total mass of particles delivered, 0.2 to 0.6 mg of the mass must be PTH wherein the therapeutically relevant dose of PTH delivered to the systemic circulation is approximately 20 micrograms to approximately 25 micrograms. In another embodiment, the dose of PTH delivered is 50 micrograms to 2 mg once per day. In another embodiment, the dose of PTH delivered 100 micrograms to 1 mg.

In another embodiment, the above-described particles have a tap density less than about 0.4 g/cm³ and/or a median geometric diameter of from between about 5 micrometers and about 30 micrometers and/or an aerodynamic diameter of from about 1 micrometer to about 5 micrometers.

The invention has numerous advantages. For example, particles suitable for inhalation can be designed to possess a controllable, in particular a rapid release profile. This rapid release profile provides for abbreviated residence of the administered bioactive agent, in particular PTH, in the lung and decreases the amount of time in which therapeutic levels of the agent are present in the local environment or systemic circulation. The rapid release of agent provides a desirable alternative to injection therapy currently used for many therapeutic agents requiring rapid release of the agent, such as PTH for the treatment of lowered PTH conditions. In addition, the invention provides a method of delivery to the pulmonary system wherein the high initial release of agent typically seen in inhalation therapy is boosted, giving very high initial release. Consequently, patient compliance and comfort can be increased by not only reducing frequency of dosing, but by providing a therapy that is more amenable to patients.

This dry powder delivery system allows for efficient and reproducible dose delivery from a small, convenient and inexpensive delivery device, including across a range of inhalation flowrates and environmental conditions. In addition, the simple and convenient inhaler together with the room temperature stable powder may offer an attractive replacement for currently available injections. This system has the potential to help achieve improved PTH levels in patients with lowered PTH conditions by increasing the willingness of patients to comply with PTH therapy.

In yet another aspect of the invention, parathyroid hormone is combined with a second active agent, in particular, an antiresorptive agent.

### BRIEF DESCRIPTION OF THE DRAWINGS

No Figures

### DETAILED DESCRIPTION OF THE INVENTION

The invention relates to particles capable of releasing bioactive agent, in particular PTH, in a rapid fashion. Methods of treating disease and delivery via the pulmonary system using these particles is also disclosed. As such, the particles possess rapid release properties. Rapid release, as that term is used herein, refers to an increased pharmacodynamic response (including, but not limited to serum levels of the bioactive agent) typically seen in the first two hours following administration, and more preferably in the first hour. Rapid release also refers to a release of active agent, in particular inhaled PTH, in which the period of release of an effective level of agent is at least the same as, preferably shorter than that seen with presently available subcutaneous injections of active agent. Active agents include but are not limited to PTH, PTH(1-84), rhPTH-(1-84) (Allelix Biopharmaceuticals), PTH fragments including but not limited to, rhPTH(1-34), teripartide(rDNA origin) recombinant parathyroid hormone (1-34) (FORTEO™, Eli Lilly & Co), hPTH (1-34), hPTH(1-31) and monocyclic hPTH (1-31) (Andreassen et al), additional PTH fragments and analogs thereof, for example, Ostabolin and Ostabolin-C™,(Zelos Therapeutics, Waltham, Mass.) as well as other C-terminal PTH fragments and N-terminal PTH fragments hereinafter collectively referred to as PTH. Preferred pharmaceutical compositions of the invention comprise hPTH 1-34, rhPTH 1-34 or Ostabolin-C, in particular the bioactive cyclic analogue (1-31) PTH fragment. Since parathyroid hormone can lead to bone resorption, bone formation, or both, it can be used to treatment options tailored to a patient's particular situation. At least part of the reason for these treatment options is the influence of parathyroid hormone on osteoclasts, osteoblasts, osteocytes and bone lining cells.

Delivery of bioactive agents to the pulmonary system, however, can result in rapid release of the agent following administration. For example, U.S. Pat. Nos. 6,080,712; 5,814,607 and 5,607,915 to Patton et al. describes the absorption of PTH34 following administration of a dry powder formulation via intratracheal delivery. The peak PTH level was reached in about 15 minutes for rats. Backström in U.S. Pat. No. 6,436,902 discloses delivering parathyroid hormone and using absorption enhancing substances to achieve the effect. Parathyroid hormone is the most important endocrine regulator of calcium and phosphorus concentration in extracellular fluid. This hormone is secreted from cells of the parathyroid glands and finds its major target cells in bone and kidney. Another hormone, parathyroid hormone-related protein, binds to the same receptor as parathyroid hormone and has major effects on development. Like most other protein hormones, parathyroid hormone is synthesized as a preprohormone. After intracellular processing, the mature hormone is packaged within the Golgi into secretory vesicles, the secreted into blood by exocytosis. Endogenous parathyroid hormone is secreted as a linear protein of 84 amino acids.

Parathyroid hormone accomplishes its job by stimulating at least three processes. First, PTH mobilized calcium from the bone. Although the mechanisms remain obscure, a well-documented effect of parathyroid hormone is to stimulate osteoclasts to reabsorb bone mineral, liberating calcium into blood. Second, PTH enhances absorption of calcium from the small intestines. Facilitating calcium absorption from the small intestine would clearly serve to elevate blood levels of calcium. Parathyroid hormone stimulates this process, but indirectly by stimulating production of the active form of vitamin D in the kidney. Vitamin D induces synthesis of a calcium-binding protein in intestinal epithelial cells that facilitates efficient absorption of calcium into blood. Third, PTH suppresses calcium loss in urine. That is, in addition to stimulating fluxes of calcium into blood from bone and intestine, parathyroid hormone puts a brake on excretion of calcium in urine, thus conserving calcium in blood. This effect is mediated by stimulating tubular reabsorption of calcium. Another effect of parathyroid hormone on the kidney is to stimulate loss of phosphate ions in urine. http://arbl.cvmbs.colostate.edu/hbooks/pathphys/endocrine/thyroid/pth.html Thus, PTH (1) increases the calcium and phosphorus release from bone, (2) decreases the loss of calcium; (3) increases the loss of phosphorus in the urine; and (4) increases the activation of 25-hydroxy vitamin D to 1,25-dihydroxy vitamin D in the kidneys. Secretion of PTH is regulated by the level of calcium in the blood. Low serum calcium causes increased PTH to be secreted, whereas increased serum calcium inhibits PTH release. Typical normal values are 10-55 pg/ml (pg/ml=picograms per milliliter.) Greater-than-normal levels of PTH may be associated with (1) chronic renal failure; (2) hyperparathyroidism; (3) malabsorption syndrome (inadequate absorption of nutrients in the intestinal tract); (4) osteomalacia in adults; (5) rickets in children; and (6) Vitamin D deficiency. Lower-than-normal levels may be associated with (1) autoimmune destruction of the parathyroid gland, (2) hypomagnesemia; (3) hypoparathyroidism; (4) metastatic bone tumor; (5) milk-alkali syndrome (excessive calcium ingestion); (6) sarcoidosis; and (7) vitamin D intoxication. There is no doubt that chronic secretion or continuous infusion of parathyroid hormone leads to decalcification of bone and loss of bone mass. However, in certain situations, treatment with parathyroid hormone can actually stimulate an increase in bone mass and bone strength. It has been found that this seemingly paradoxical effect occurs when the hormone is administered in pulses (e.g. by once daily injection), and such treatment appears to be an effective therapy for diseases such as osteoporosis. Thus, PTH is useful for the treatment of subjects with lower-than-normal levels of parathyroid hormone. PTH may be used in the treatment of osteonecrosis in patients receiving protease inhibitors for other conditions.

In one embodiment, the rapid release particles are formulated using PTH, sodium citrate and a phospholipid. In another embodiment, the particles are formulated using PTH, leucine and a phospholipid. It is believed that the selection of the appropriate phospholipid affects the release profile as described in more detail below. In a preferred embodiment, the rapid release is characterized by both the period of release being shorter and the levels of agent released being greater.

The particles of the invention have specific drug release properties. Release rates can be controlled as described below and as further described in U.S. application Ser. No. 09/644,736 filed Aug. 23, 2000 entitled “Modulation of Release from Dry Powder Formulations” by Sujit Basu, et al. Drug release rates can be described in terms of the half-time of release of a bioactive agent from a formulation. As used herein the term “half-time” refers to the time required to release 50% of the initial drug payload contained in the particles. Fast or rapid drug release rates generally are less than 30 minutes and range from about 1 minute to about 60 minutes.

Drug release rates can also be described in terms of release constants which are known to those skilled in the art. Release rates of drugs from particles can be controlled or optimized by adjusting the thermal properties or physical state transitions of the particles. The particles of the invention can be characterized by their matrix transition temperature. As used herein, the term “matrix transition temperature” refers to the temperature at which particles are transformed from glassy or rigid phase with less molecular mobility to a more amorphous, rubbery or molten state or fluid-like phase. As used herein, “matrix transition temperature” is the temperature at which the structural integrity of a particle is diminished in a manner which imparts faster release of drug from the particle. Above the matrix transition temperature, the particle structure changes so that mobility of the drug molecules increases resulting in faster release. In contrast, below the matrix transition temperature, the mobility of the drug particles is limited, resulting in a slower release. The “matrix transition temperature” can relate to different phase transition temperatures, for example, melting temperature (Tₘ), crystallization temperature (T_{c}) and glass transition temperature (T_{g}) which represent changes of order and/or molecular mobility within solids. The term “matrix transition temperature,” as used herein, refers to the composite or main transition temperature of the particle matrix above which release of drug is faster than below.

Experimentally, matrix transition temperatures can be determined by methods known in the art, in particular by differential scanning calorimetry (DSC). Other techniques to characterize the matrix transition behavior of particles or dry powders include synchrotron X-ray diffraction and freeze fracture electron microscopy.

Matrix transition temperatures can be employed to fabricate particles having desired drug release kinetics and to optimize particle formulations for a desired drug release rate. Particles having a specified matrix transition temperature can be prepared and tested for drug release properties by in vitro or in vivo release assays, pharmacokinetic studies and other techniques known in the art. Once a relationship between matrix transition temperatures and drug release rates is established, desired or targeted release rates can be obtained by forming and delivering particles which have the corresponding matrix transition temperature. Drug release rates can be modified or optimized by adjusting the matrix transition temperature of the particles being administered.

The particles of the invention include one or more materials which, alone or in combination, promote or impart to the particles a matrix transition temperature that yields a desired or targeted drug release rate. Properties and examples of suitable materials or combinations thereof are further described below. For example, to obtain a rapid release of a drug, materials, which, when combined, result in low matrix transition temperatures, are preferred. As used herein, “low transition temperature” refers to particles which have a matrix transition temperature which is below or about the physiological temperature of a subject. Particles possessing low transition temperatures tend to have limited structural integrity and be more amorphous, rubbery, in a molten state, or fluid-like. Without wishing to be held to any particular interpretation of a mechanism of action, it is believed that, for particles having low matrix transition temperatures, the integrity of the particle matrix undergoes transition within a short period of time when exposed to body temperature (typically around 37° C.) and high humidity (approaching 100% in the lungs) and that the components of these particles tend to possess high molecular mobility allowing the drug to be quickly released and available for uptake.

Designing and fabricating particles with a mixture of materials having high phase transition temperatures can be employed to modulate or adjust matrix transition temperatures of resulting particles and corresponding release profiles for a given drug.

Combining appropriate amounts of materials to produce particles having a desired transition temperature can be determined experimentally, for example, by forming particles having varying proportions of the desired materials, measuring the matrix transition temperatures of the mixtures (for example, by DSC), selecting the combination having the desired matrix transition temperature and, optionally, further optimizing the proportions of the materials employed.

The amounts of phospholipids to be used to form particles having a desired or targeted matrix transition temperature can be determined experimentally, for example, by forming mixtures in various proportions of the phospholipids of interest, measuring the transition temperature for each mixture, and selecting the mixture having the targeted transition temperature. The effects of phospholipid miscibility on the matrix transition temperature of the phospholipid mixture can be determined by combining a first phospholipid with other phospholipids having varying miscibilities with the first phospholipid and measuring the transition temperature of the combinations. In a preferred embodiment, the particles include one or more phospholipids. The phospholipid or combination of phospholipids is selected to impart specific drug release properties to the particles. Phospholipids suitable for pulmonary delivery to a human subject are preferred. In one embodiment, the phospholipid is endogenous to the lung. In another embodiment, the phospholipid is non-endogenous to the lung. The phospholipid can be present in the particles in an amount ranging from about 1 to about 99 weight %. Preferably, it can be present in the particles in an amount ranging from about 10 to about 80 weight %. In other example, the amount of phospholipid in the particles is approximately 40% to 80%, for example, 35%, 40%, 45%, 50%, 55%, 60%, 65%, 70%, 75%, 80% or 85%. In another example, the phospholipid is DPPC. Examples of phospholipids include, but are not limited to, phosphatidic acids, phosphatidylcholines, phosphatidylethanolamines, phosphatidylglycerols, phosphatidylserines, phosphatidylinositols or a combination thereof. Modified phospholipids, for example, phospholipids having their head group modified, e.g., alkylated or polyethylene glycol (PEG)-modified, also can be employed.

Combinations of one or more phospholipids with other materials also can be employed to achieve a desired matrix transition temperature. Examples include polymers and other biomaterials, such as, for instance, lipids, sphingolipids, cholesterol, surfactants, polyaminoacids, polysaccharides, proteins, salts and others. Amounts and miscibility parameters selected to obtain a desired or targeted matrix transition temperatures can be determined.

In general, phospholipids, combinations of phospholipids, as well as combinations of phospholipids with other materials, which yield a matrix transition temperature no greater than about the physiological body temperature of a patient, are preferred in fabricating particles which have fast drug release properties. Such phospholipids or phospholipid combinations are referred to herein as having low transition temperatures. Examples of suitable low transition temperature phospholipids are listed in Table 1. Transition temperatures shown are obtained from the Avanti Polar Lipids (Alabaster, Ala.) Catalog.

TABLE 1

| | | Transition |
|---|---|---|
| | Phospholipids | Temperature |
| 1 | 1,2-Dilauroyl-sn-glycero-3- | −1° C. |
| | phosphocholine (DLPC) | |
| 2 | 1,2-Ditridecanoyl-sn-glycero- | 14° C. |
| | 3-phosphocholine | |
| 3 | 1,2-Dimyristoyl-sn-glycero-3- | 23° C. |
| | phosphocholine (DMPC) | |
| 4 | 1,2-Dipentadecanoyl-sn-glycero- | 33° C. |
| | 3-phosphocholine | |
| 5 | 1,2-Dipalmitoyl-sn-glycero- | 41° C. |
| | 3-phosphocholine (DPPC) | |
| 6 | 1-Myristoyl-2-palmitoyl-sn- | 35° C. |
| | glycero-3-phosphocholine | |
| 7 | 1-Myristoyl-2-stearoyl-sn- | 40° C. |
| | glycero-3-phosphocholine | |
| 8 | 1-Palmitoyl-2-myristoyl-sn- | 27° C. |
| | glycero-3-phosphocholine | |
| 9 | 1-Stearoyl-2-myristoyl-sn- | 30° C. |
| | glycero-3-phosphocholine | |
| 10 | 1,2-Dilauroyl-sn-glycero- | 31° C. |
| | 3-phosphate (DLPA) | |
| 11 | 1,2-Dimyristoyl-sn-glycero- | 35° C. |
| | 3-[phospho-L-serine] | |
| 12 | 1,2-Dimyristoyl-sn-glycero- | 23° C. |
| | 3-[phospho-rac-(1-glycerol)](DMPG) | |
| 13 | 1,2-Dipalmitoyl-sn-glycero- | 41° C. |
| | 3-[phospho-rac-(1-glycerol)] (DPPG) | |
| 14 | 1,2-Dilauroyl-sn-glycero-3- | 29° C. |
| | phosphoethanolamine (DLPE) | |

Phospholipids having a head group selected from those found endogenously in the lung, e.g., phosphatidylcholine, phosphatidylethanolamines, phosphatidylglycerols, phosphatidylserines, phosphatidylinositols or a combination thereof are preferred.

The above materials can be used alone or in combinations. Other phospholipids which have a phase transition temperature no greater than a patient's body temperature, also can be employed, either alone or in combination with other phospholipids or materials.

The particles of the instant invention, in particular the rapid release particles, are delivered pulmonarily. “Pulmonary delivery,” as that term is used herein refers to delivery to the respiratory tract. The “respiratory tract,” as defined herein, encompasses the upper airways, including the oropharynx and larynx, followed by the lower airways, which include the trachea followed by bifurcations into the bronchi and bronchioli (e.g., terminal and respiratory). The upper and lower airways are called the conducting airways. The terminal bronchioli then divide into respiratory bronchioli which then lead to the ultimate respiratory zone, namely, the alveoli, or deep lung. The deep lung, or alveoli, are typically the desired target of inhaled therapeutic formulations for systemic drug delivery.

“Pulmonary pH range,” as that term is used herein, refers to the pH range which can be encountered in the lung of a patient. Typically, in humans, this range of pH is from about 6.4 to about 7.0, such as from 6.4 to about 6.7. pH values of the airway lining fluid (ALF) have been reported in “Comparative Biology of the Normal Lung”, CRC Press, (1991) by R. A. Parent and range from 6.44 to 6.74.

Therapeutic or prophylactic agents, can also be referred to herein as “bioactive agents,” “medicaments” or “drugs.” The amount of therapeutic or prophylactic agent present in the particles can range from about 0.1 weight % to about 30 weight percent. In other embodiments, the amount of bioactive agent in the particles is approximately 10% to 20%, for example, 5%, 10%, 15%, 20%, 25%, 30% or amounts in between.

Combinations of bioactive agents also can be employed. Particles in which the drug is distributed throughout a particle are preferred. Additional suitable bioactive agents include agents which can act locally, systemically or a combination thereof. The term “bioactive agent” as used herein, is an agent, or its pharmaceutically acceptable salt, which when released in vivo, possesses the desired biological activity, for example, therapeutic and/or prophylactic properties in vivo. The agents can have a variety of biological activities, such as antiresporptive agents including but not limited to the estrogens; selective estrogen receptor modulators (SERM), raloxifene (Evista, Eli Lilly); and calcitonin, in particular, nasal salmon calcitonin (Miacalcin, Novartis) and bisphosphonates, such as alendronate (Fosamax, Merck) and risedronate (Actonel, Procter & Gambel/Aventis). Preferred doses of the antiresorptive agent, in particular, alendronate, ranges from about 5-25 mg per day, in particular, 10 mg daily. It is expected that such a combination will enhance the effect on bone mineral density.

The particles can further comprise a carboxylic acid which is distinct from the agent and lipid, in particular a phospholipid. In one embodiment, the carboxylic acid includes at least two carboxyl groups. Carboxylic acids, include the salts thereof as well as combinations of two or more carboxylic acids and/or salts thereof. In a preferred embodiment, the carboxylic acid is a hydrophilic carboxylic acid or salt thereof. Suitable carboxylic acids include but are not limited to hydroxydicarboxylic acids, hydroxytricarboxylic acids and the like. Citric acid and citrates, such as, for example, sodium citrate, are preferred. Combinations or mixtures of carboxylic acids and/or their salts also can be employed.

The carboxylic acid can be present in the particles in an amount ranging from about 0 weight % to about 80 weight %. Preferably, the carboxylic acid can be present in the particles in an amount of about 10% to about 20%, for example 5%, 10%, 15%, 20%, or 25%.

The particles suitable for use in the invention can further comprise an amino acid. Particles containing amino acids are disclosed in U.S. Pat. No. 6,586,000 issued Jul. 1, 2003 and in co-pending U.S. application Ser. No. 09/644,320 filed Aug. 23, 2000 the entire teachings of both are incorporated herein by reference. In a preferred embodiment the amino acid is hydrophobic. Examples of amino acids which can be employed include, but are not limited to: glycine, proline, alanine, cysteine, methionine, valine, leucine, tyrosine, isoleucine, phenylalanine, tryptophan. Preferred hydrophobic amino acids include leucine, isoleucine, alanine, valine, phenylalanine, glycine and tryptophan. Combinations of hydrophobic amino acids can also be employed. Furthermore, combinations of hydrophobic and hydrophilic (preferentially partitioning in water) amino acids, where the overall combination is hydrophobic, can also be employed. Combinations of one or more amino acids can also be employed. Non-naturally occurring amino acids include, for example, beta-amino acids. Both D, L configurations and racemic mixtures of hydrophobic amino acids can be employed. Suitable hydrophobic amino acids can also include amino acid derivatives or analogs. As used herein, an amino acid analog includes the D or L configuration of an amino acid having the following formula: —NH—CHR—CO—, wherein R is an aliphatic group, a substituted aliphatic group, a benzyl group, a substituted benzyl group, an aromatic group or a substituted aromatic group and wherein R does not correspond to the side chain of a naturally-occurring amino acid. As used herein, aliphatic groups include straight chained, branched or cyclic C1-C8 hydrocarbons which are completely saturated, which contain one or two heteroatoms such as nitrogen, oxygen or sulfur and/or which contain one or more units of unsaturation. Aromatic or aryl groups include carbocyclic aromatic groups such as phenyl and naphthyl and heterocyclic aromatic groups such as imidazolyl, indolyl, thienyl, furanyl, pyridyl, pyranyl, oxazolyl, benzothienyl, benzofuranyl, quinolinyl, isoquinolinyl and acridintyl. When speaking of an amino acid, it is understood that any of the above can be employed.

A number of the suitable amino acids, amino acids analogs and salts thereof can be obtained commercially. Others can be synthesized by methods known in the art. Synthetic techniques are described, for example, in Green and Wuts, “Protecting Groups in Organic Synthesis,” John Wiley and Sons, Chapters 5 and 7, 1991. Hydrophobicity is generally defined with respect to the partition of an amino acid between a nonpolar solvent and water. Hydrophobic amino acids are those acids which show a preference for the nonpolar solvent. Relative hydrophobicity of amino acids can be expressed on a hydrophobicity scale on which glycine has the value 0.5. On such a scale, amino acids which have a preference for water have values below 0.5 and those that have a preference for nonpolar solvents have a value above 0.5. As used herein, the term hydrophobic amino acid refers to an amino acid that, on the hydrophobicity scale has a value greater or equal to 0.5, in other words, has a tendency to partition in the nonpolar acid which is at least equal to that of glycine.

The amino acid can be present in the particles of the invention in an amount from about 5 weight % to about 90 weight %. Preferably, the amino acid can be present in the particles in an amount ranging from about 15 weight % to about 50 weight %. The salt of a hydrophobic amino acid can be present in the particles. Methods of forming and delivering particles which include an amino acid are described in U.S. patent application Ser. No. 09/382,959, filed on Aug. 25, 1999, entitled “Use of Simple Amino Acids to Form Porous Particles During Spray Drying”, and U.S. patent application Ser. No. 09/644,320, filed on Aug. 23, 2000, entitled “Use of Simple Amino Acids to Form Porous Particles”, the entire teachings of which are incorporated herein by reference.

In a further embodiment, the particles can also include other materials such as, for example, buffer salts, proteins, peptides, polypeptides, fatty acids, fatty acid esters, inorganic compounds, and phosphates. However, preferred particles are free of albumin, dextran, polysaccharides, lactose, trehalose, and cyclodextrins.

In yet another embodiment, the particles include a surfactant other than one of the charged lipids described above. As used herein, the term “surfactant” refers to any agent which preferentially absorbs to an interface between two immiscible phases, such as the interface between water and an organic polymer solution, a water/air interface or organic solvent/air interface. Surfactants generally possess a hydrophilic moiety and a lipophilic moiety, such that, upon absorbing toparticles, they tend to present moieties to the external environment that do not attract similarly-coated particles, thus reducing particle agglomeration. Surfactants may also promote absorption of a therapeutic or prophylactic agent and increase bioavailability of the agent.

Suitable surfactants which can be employed in fabricating the particles of the invention include but are not limited to hexadecanol; fatty alcohols such as polyethylene glycol (PEG); polyoxyethylene-9-lauryl ether; a surface active fatty acid, such as palmitic acid or oleic acid; glycocholate; surfactin; a poloxomer; a sorbitan fatty acid ester such as sorbitan trioleate (Span 85); and tyloxapol.

The surfactant can be present in the particles in an amount ranging from about 9 weight % to about 94 weight %. Preferably, when in the presence of citrate, it can be present in the particles in an amount ranging from about 60 weight % to about 94 weight %. Preferably, when in the presence of citrate and leucine, it can be present in an amount ranging from about 1 weight % and from about 89 weight %.

It is understood that when the particles includes a carboxylic acid, a multivalent salt, an amino acid, a surfactant or any combination thereof that interaction between these components of the particle and the charged lipid can occur.

The particles, also referred to herein as powder, can be in the form of a dry powder suitable for inhalation. In a particular embodiment, the particles can have a tap density of less than about 0.4 g/cm³. Particles which have a tap density of less than about 0.4 g/cm³ (e.g., 0.4 g/cm³) are referred to herein as “aerodynamically light particles”. More preferred are particles having a tap density less than about 0.1 g/cm³ (e.g., 0.1 g/cm³).

Aerodynamically light particles have a preferred size, e.g., a volume median geometric diameter (VMGD) of at least about 5 microns (μm). In one embodiment, the VMGD is from about 5 μm to about 30 μm (for example, 5, 10, 15, 20, 25 or 30 μm). In another embodiment of the invention, the particles have a VMGD ranging from about 9 μm to about 30 μm. In other embodiments, the particles have a median diameter, mass median diameter (MMD), a mass median envelope diameter (MMED) or a mass median geometric diameter (MMGD) of at least 5 μm, for example, from about 5 μm to about 30 μm (for example, 5, 10, 15, 20, 25 or 30 μm), or from about 7 μm to about 8 μm (for example, 6 μm, 7 μm, or 8 μm).

Aerodynamically light particles preferably have “mass median aerodynamic diameter” (MMAD), also referred to herein as “aerodynamic diameter”, between about 1 μm and about 5 μm (for example 1, 2, 3, 4, or 5 μm). In one embodiment of the invention, the MMAD is between about 1 μm and about 3 μm. In another embodiment, the MMAD is between about 3 μm and about 5 μm.

In another embodiment of the invention, the particles have an envelope mass density, also referred to herein as “mass density” of less than about 0.4 g/cm³. The envelope mass density of an isotropic particle is defined as the mass of the particle divided by the minimum sphere envelope volume within which it can be enclosed.

Tap density can be measured by using instruments known to those skilled in the art. Features which can contribute to low tap density include irregular surface texture and porous structure. Indeed, high surface area particles such as those described in co-pending application U.S. Ser. No. 10/300,657 entitled “Improved Particulate Compositions for Pulmonary Delivery,” Batycky et al. are particularly suitable for use in the invention. The entire teachings of U.S. Ser. No. 10/300,657 are incorporated herein by reference.

The diameter of the particles, for example, their VMGD, can be measured using instruments well known in the art. The diameter of particles in a sample will range depending upon factors such as particle composition and methods of synthesis. The distribution of size of particles in a sample can be selected to permit optimal deposition within targeted sites within the respiratory tract.

Experimentally, aerodynamic diameter can be determined using method well know to those in the art. See co-pending patent application U.S. patent application Ser. No. 10/179,463 entitled “Particles for Inhalation Having Rapid Release Properties” Schmitke et al. and U.S. patent application entitled, “High Efficient Delivery of a Large Therapeutic Mass Aerosol,” application Ser. No. 09/591,307, filed Jun. 9, 2000, and continuation-in-part of U.S. patent application Ser. No. 09/878,146, entitled, “Highly Efficient Delivery of a Large Therapeutic Mass Aerosol,” filed Jun. 8, 2001. Edwards et al. all of the entire teachings of which are incorporated herein by reference.

Particles which have a tap density less than about 0.4 g/cm³, median diameters of at least about 5 μm, and an aerodynamic diameter of between about 1 μm and about 5 μm, preferably between about 1 μm and about 3 μm, are more capable of escaping inertial and gravitational deposition in the oropharyngeal region, and are targeted to the airways or the deep lung. The use of larger, more porous particles is advantageous since they are able to aerosolize more efficiently than smaller, denser aerosol particles such as those currently used for inhalation therapies.

The particles may be fabricated with the appropriate material, surface roughness, diameter and tap density for localized delivery to selected regions of the respiratory 2 5 tract such as the deep lung or upper or central airways. For example, higher density or larger particles may be used for upper airway delivery, or a mixture of varying sized particles in a sample, provided with the same or different therapeutic agent may be administered to target different regions of the lung in one administration. Particles having an aerodynamic diameter ranging from about 3 to about 5 μm are preferred for delivery to the central and upper airways. Particles having an aerodynamic diameter ranging from about 1 to about 3 μm are preferred for delivery to the deep lung.

In one embodiment, particles of the instant invention have an aerodynamic diameter from about 1 to about 3 microns and a mean geometric diameter at 2 bar/16 mbar pressure of between about 5 microns and about 7.5 microns. In another embodiment, particles have about 35-55% of the particles with a fine particle fraction (FPF) less than about 3.4 microns, as detected using a 2 stage Anderson Cascade Impactor (ACI) assay. In another embodiment, particles have about 60-80% of the particles with a fine particle fraction of less than about 5.6 microns. Methods of measuring fine particle fraction using a 2 stage ACI assay are well known to those skilled in the art. One example of such an assay is as follows. Fine Particle Fractions (FPF) are measured using a reduced Thermo Anderson Cascade Impactor with two stages. Ten milligrams of powder are weighed into a size 2 hydroxpropyl methyl cellulose (HPMC) capsule. The powders are dispersed using a single-step, breath-actuated dry powder inhaler operated at 60 L/min for 2 seconds. The stages are selected to collect particles of an effective cutoff diameter (ECD) of (1) between 5.6 microns and 3.4 microns and (2) less than 3.4 microns and are fitted with porous filter material to collect the powder deposited. The mass deposited on each stage is determined gravimetrically. FPF is then expressed as a fraction of the total mass loaded into the capsule.

In another embodiment, particles of the instant invention have a mean geometric diameter at 1 bar of about 5 to about 8 microns as determined by RODOS.

Inertial impaction and gravitational settling of aerosols are predominant deposition mechanisms in the airways and acini of the lungs during normal breathing conditions. Edwards, D.A., J. Aerosol Sci., 26:293-317 (1995). The importance of both deposition mechanisms increases in proportion to the mass of aerosols and not to particle (or envelope) volume. Since the site of aerosol deposition in the lungs is determined by the mass of the aerosol (at least for particles of mean aerodynamic diameter greater than approximately 1 μm), diminishing the tap density by increasing particle surface irregularities and particle porosity permits the delivery of larger particle envelope volumes into the lungs, all other physical parameters being equal.

Suitable particles can be fabricated or separated, for example, by filtration or centrifugation, to provide a particle sample with a preselected size distribution. For example, greater than about 30%, 50%, 70%, or 80% of the particles in a sample can have a diameter within a selected range of at least about 5 μm. The selected range within which a certain percentage of the particles must fall may be for example, between about 5 and about 30 μm, or optimally between about 5 and about 15 μm. In one preferred embodiment, at least a portion of the particles have a diameter between about 9 and about 11 μm. Optionally, the particle sample also can be fabricated wherein at least about 90%, or optionally about 95% or about 99%, have a diameter within the selected range. The presence of the higher proportion of the aerodynamically light, larger diameter particles in the particle sample enhances the delivery of therapeutic or diagnostic agents incorporated therein to the deep lung. Large diameter particles generally mean particles having a median geometric diameter of at least about 5 μm.

The particles can be prepared by spray drying. For example, a spray drying mixture, also referred to herein as “feed solution” or “feed mixture”, which includes the bioactive agent and one or more charged lipids having a charge opposite to that of the active agent upon association are fed to a spray dryer. For example, when employing a protein active agent, the agent may be dissolved in a buffer system above or below the pI of the agent. Specifically, PTH, for example, may be dissolved in an aqueous buffer system (e.g., citrate, phosphate, acetate, etc.) or in 0.01 N HCl. The pH of the resultant solution then can be adjusted to a desired value using an appropriate base solution (e.g., 1 N NaOH). In one preferred embodiment, the pH may be adjusted to about pH 7.4. In another embodiment, the pH may be adjusted to about pH 4.0. In addition, if desired the solutions can be heated to temperatures below their boiling points, for example, approximately 50° C. Typically the cationic phospholipid is dissolved in an organic solvent or combination of solvents. The two solutions are then mixed together and the resulting mixture is spray dried.

Suitable organic solvents that can be present in the mixture being spray dried include, but are not limited to, alcohols, for example, ethanol, methanol, propanol, isopropanol, butanols, and others. Other organic solvents include, but are not limited to, perfluorocarbons, dichloromethane, chloroform, ether, ethyl acetate, methyl tert-butyl ether and others. Aqueous solvents that can be present in the feed mixture include water and buffered solutions. Both organic and aqueous solvents can be present in the spray-drying mixture fed to the spray dryer. In one embodiment, an ethanol water solvent is preferred with the ethanol:water ratio ranging from about 50:50 to about 90:10. The mixture can have a neutral, acidic or alkaline pH. Optionally, a pH buffer can be included. Preferably, the pH can range from about 3 to about 10.

The total amount of solvent or solvents being employed in the mixture being spray dried generally is greater than about 98 weight percent. The amount of solids (drug, charged lipid and other ingredients) present in the mixture being spray dried can vary from about 1.0 weight percent to about 1.5 weight percent. Using a mixture which includes an organic and an aqueous solvent in the spray drying process allows for the combination of hydrophilic and hydrophobic components, while not requiring the formation of liposomes or other structures or complexes to facilitate solubilization of the combination of such components within the particles.

Suitable spray-drying techniques are described, for example, by K. Masters In “Spray Drying Handbook,” John Wiley & Sons, New York, 1984. Generally, during spray-drying, heat from a hot gas such as heated air or nitrogen is used to evaporate the solvent from droplets formed by atomizing a continuous liquid feed. Other spray-drying techniques are well known to those skilled in the art. In a preferred embodiment, a rotary atomizer is employed. An example of a suitable spray dryer using rotary atomization includes the Mobile Minor spray dryer, manufactured by Niro, Denmark. The hot gas can be, for example, air, nitrogen or argon.

Preferably, the particles of the invention are obtained by spray drying using an inlet temperature between about 100° C. and about 400° C. and an outlet temperature between about 50° C. and about 130° C. The spray dried particles can be fabricated with a rough surface texture to reduce particle agglomeration and improve flowability of the powder. The spray-dried particle can be fabricated with features which enhance aerosolization via dry powder inhaler devices, and lead to lower deposition in the mouth, throat and inhaler device.

The particles of the invention can be employed in compositions suitable for drug delivery via the pulmonary system. For example, such compositions can include the particles and a pharmaceutically acceptable carrier for administration to a patient, preferably for administration via inhalation. The particles can be co-delivered with other similarly manufactured particles that may or may not contain yet another drug. Methods for co-delivery of particles is disclosed in U.S. patent application Ser. No. 09/878,146, filed Jun. 8, 2001, the entire teachings of which are incorporated herein by reference. The particles can also be co-delivered with larger carrier particles, not including a therapeutic agent, the latter possessing mass median diameters, for example, in the range between about 50 μm and about 100 μm. The particles can be administered alone or in any appropriate pharmaceutically acceptable carrier, such as a liquid, for example, saline, or a powder, for administration to the respiratory system. Particles including a medicament, for example, one or more of drugs, are administered to the respiratory tract of a patient in need of treatment. Administration of particles to the respiratory system can be by means such as those known in the art. For example, particles are delivered from an inhalation device. In a preferred embodiment, particles are administered via a dry powder inhaler (DPI). Metered-dose-inhalers (MDI), nebulizers or instillation techniques also can be employed.

A variety of suitable devices and methods of inhalation which can be used to administer particles to a patient's respiratory tract are known in the art. For example, suitable inhalers are described in U.S. Pat. No. 4,069,819, issued Aug. 5, 1976 to Valentini, et al., U.S. Pat. No. 4,995,385 issued Feb. 26, 1991 to Valentini, et al., and U.S. Pat. No. 5,997,848 issued Dec. 7, 1999 to Patton, et al. Other examples include, but are not limited to, the Spinhaler® (Fisons, Loughborough, U.K.), Rotahaler® (Glaxo-Wellcome, Research Triangle Technology Park, N.C.), FlowCaps®(Hovione, Loures, Portugal), Inhalator® (Boehringer-Ingelheim, Germany), and the Aerolizer® (Novartis, Switzerland), the diskhaler (Glaxo-Wellcome, RTP, NC) and others, such as those known to those skilled in the art. Preferably, the particles are administered as a dry powder via a dry powder inhaler.

In one embodiment, the dry powder inhaler is a simple, breath actuated device. An example of a suitable inhaler which can be employed is described in U.S. patent application, entitled “Inhalation Device and Method”, by David A. Edwards, et al., filed on Apr. 16, 2001 under Attorney Docket No. 00166.0109.US00. The entire contents of this application are incorporated by reference herein. This pulmonary delivery system is particularly suitable because it enables efficient dry powder delivery of small molecules, proteins and peptide drug particles deep into the lung. Particularly suitable for delivery are the unique porous particles, such as the PTH particles described herein, which are formulated with a low mass density, relatively large geometric diameter and optimum aerodynamic characteristics (Edwards et al., 1998). These particles can be dispersed and inhaled efficiently with a simple inhaler device, as low forces of cohesion allow the particles to deaggregate easily. In particular, the unique properties of these particles confers the capability of being simultaneously dispersed and inhaled.

In one embodiment, the volume of the receptacle is at least about 0.37 cm³. In another embodiment, the volume of the receptacle is at least about 0.48 cm³. In yet another embodiment, are receptacles having a volume of at least about 0.67 cm³ or 0.95 cm³. The invention is also drawn to receptacles which are capsules, for example, capsules designated with a particular capsule size, such as 2, 1, 0, 00 or 000. Suitable capsules can be obtained, for example, from Shionogi (Rockville, Md.). Blisters can be obtained, for example, from Hueck Foils, (Wall, N.J.). Other receptacles and other volumes thereof suitable for use in the instant invention are known to those skilled in the art.

The receptacle encloses or stores particles and/or respirable compositions comprising particles. In one embodiment, the particles and/or respirable compositions comprising particles are in the form of a powder. The receptacle is filled with particles and/or compositions comprising particles, as known in the art. For example, vacuum filling or tamping technologies may be used. Generally, filling the receptacle with powder can be carried out by methods known in the art. In one embodiment of the invention, the particles which are enclosed or stored in a receptacle have a mass of at least about 5 milligrams. In another embodiment, the mass of the particles stored or enclosed in the receptacle comprises a mass of bioactive agent from at least about 1.5 mg to at least about 20 milligrams.

Preferably, particles administered to the respiratory tract travel through the upper airways (oropharynx and larynx), the lower airways, which include the trachea followed by bifurcations into the bronchi and bronchioli and through the terminal bronchioli which in turn divide into respiratory bronchioli leading then to the ultimate respiratory zone, the alveoli or the deep lung. In a preferred embodiment of the invention, most of the mass of particles deposits in the deep lung. In another embodiment of the invention, delivery is primarily to the central airways. Delivery to the upper airways can also be obtained.

In one embodiment of the invention, delivery to the pulmonary system of particles is in a single, breath-actuated step, as described in U.S. patent application entitled, “High Efficient Delivery of a Large Therapeutic Mass Aerosol,” U.S. patent application Ser. No. 09/591,307, filed Jun. 9, 2000, and continuation-in-part of U.S. patent application Ser. No. 09/878,146, entitled, “Highly Efficient Delivery of a Large Therapeutic Mass Aerosol,” filed Jun. 8, 2001, the entire teachings of which are incorporated herein by reference. In one embodiment, the dispersing and inhalation occurs simultaneously in a single inhalation in a breath-actuated device. An example of a suitable inhaler which can be employed is described in U.S. patent application, entitled “Inhalation Device and Method,” by David A. Edwards, et al., filed on Apr. 16, 2001 under Attorney Docket No. 00166.0109.US00. The entire contents of this application are incorporated by reference herein. In another embodiment of the invention, at least 50% of the mass of the particles stored in the inhaler receptacle is delivered to a subject's respiratory system in a single, breath-activated step.

In one further embodiment, at least 1.5 milligrams, or at least 5 milligrams, or at least 10 milligrams of a particles contained bioactive agent is delivered by administering, in a single breath, to a subject's respiratory tract particles enclosed in the receptacle. Amounts of bioactive agent in the particles ranges from about 50 micrograms to about 2 mg.

As used herein, the term “effective amount” means the amount needed to achieve the desired therapeutic or diagnostic effect or efficacy. The actual effective amounts of drug can vary according to the specific drug or combination thereof being utilized, the particular composition formulated, the mode of administration, and the age, weight, condition of the patient, and severity of the symptoms or condition being treated. Dosages for a particular patient can be determined by one of ordinary skill in the art using conventional considerations (e.g., by means of an appropriate, conventional pharmacological protocol.) See for example “Anabolic effect of low doses of a fragment of human parathyroid hormone on the skeleton in postmenopausal osteoporosis” Reeve J., Lancet.;1(7968):1035-8 (1976) and “Effect of parathyroid hormone (1-34) on fractures and bone mineral density in post menopausal women with osteoporosis”, Neer R. M., NEJM 344(19):1434-41 (2001).

Aerosol dosage, formulations and delivery systems also may be selected for a particular therapeutic application, as described, for example, In Gonda, I. “Aerosols for delivery of therapeutic and diagnostic agents to the respiratory tract,” in Critical Reviews in Therapeutic Drug Carrier Systems, 6:273-313, 1990; and In Moren, “Aerosol dosage forms and formulations,” in: Aerosols in Medicine. Principles, Diagnosis and Therapy, Moren, et al., Eds, Esevier, Amsterdam, 1985.

As used herein, the term “a” or “an” refers to one or more.

The term “nominal dose” as used herein, refers to the total mass of bioactive agent which is present in the mass of particles targeted for administration and represents the maximum amount of bioactive agent available for administration.

Applicants'technology is based upon pulmonary delivery of dry powder aerosols 2 5 composed of large, porous particles wherein each individual particle is capable of comprising both drug and excipient within a porous matrix. The particles are geometrically large but have low mass density and aerodynamic size. This results in a powder that is easily dispersible. The ease of dispersibility of the dry powder aerosols of large porous particles described herein allows for efficient systemic delivery of 3 0 protein therapeutics from simple, breath activated, capsule based inhalers.

The invention also features a kit comprising at least two receptacles, each receptacle containing a different amount of dry powder PTH suitable for inhalation. The powder can be, but is not limited to any such dry powder PTH as described herein. In addition, the invention also features a kit comprising two or more receptacles comprising two or more unit dosages comprising particles comprising the bioactive agent formulations described herein. Depending on the bioavailability of the bioactive agent in the formulation, the formulation can contain more bioactive agent than the amount that is delivered to the subject's bloodstream.

The kits described herein can be used to deliver bioactive agents, for example, PTH to a subject in need of the bioactive agent. When the bioactive agent is PTH, the dose administered to the subject can be altered, for example, by a patient or by a medical provider, by increasing or decreasing the number of receptacles (e.g., capsules) of PTH containing particles, thereby increasing or decreasing the unit dosage of the PTH. When a patient is in need of a higher dose of PTH than usual, that patient can administer to himself or herself additional receptacles, or a different combination of receptacles, so that the dose of PTH is increased to the desired amount. Conversely, when a patient needs less PTH, the patient can administer to himself or herself fewer receptacles, or a different combination of receptacles, such that the dose is decreased to the desired amount. The kits may also contain instructions for the use of the reagents in the kits (e.g., the receptacles containing the formulation). Through the use of such kits, accurate dosing can be accomplished.

Methods of the instant invention are employed when a subject has been diagnosed as needing parathyroid hormone. One method of determining this need is testing serum calcium levels and PTH levels.

### EXEMPLIFICATION

### EXAMPLE 1

The following example describes the preparation of particles with a 15 wt % PTH load (DPPC/ PTH/ citrate, 75/15/10 wt %). The following procedure details preparation of a one liter solution batch. An aqueous solution is prepared as follows. 0.4 L of a pH 2.5 citrate buffer is prepared by dissolving 1.26 gr of citric acid monohydrate in 0.4 L of sterile water for injection and adjusting the pH to 2.5 with 1.0N HCl. 2.25 gr of PTH are then dissolved into this citrate buffer. Finally, 1.0 N sodium hydroxide (NaOH) is added until the pH had been adjusted to 6.7. An organic solution is prepared by dissolving 11.25 g DPPC in 600 mL of ethanol (200 proof, USP).

Prior to spray drying, both the aqueous and organic solutions are in-line filtered (0.22 micron filter) and then in-line heated to 50° C. A spray-drying feed solution is prepared by in-line static mixing the heated aqueous solution with the heated organic solution. The resulting aqueous/organic feed solution is combined such that it has a final volumetric composition of 60% ethanol/40% water with a solute concentration of 15 gr/L. This feed solution is pumped at a controlled rate of 50 mL/min into the top of the spray-drying chamber (Size 1 Niro spray dryer, Model Mobil Minor 2000). Upon entering the spray-drying chamber, the solution is atomized into small droplets of liquid using a 2 fluid atomizer (Liquid Cap 2850 and Gas Cap 67147, Spraying Systems Inc) with an atomization gas rate of 62 g/min. The process gas, heated dry nitrogen, is introduced at a controlled rate of 110 kg/hr into the top of the drying chamber. As the liquid droplets contact the heated nitrogen, the liquid evaporates and porous particles are formed. The temperature of the inlet drying gas is 128° C. and the outlet process gas temperature is 67.5° C. The particles exit the drying chamber with the process gas and enters a product filter downstream. The product filter separates the porous particles from the process gas stream. The process gas exits from the top of the collector and is directed to the exhaust system. Periodically, the filter is reverse pulsed and product exits from the bottom of the product filter and is recovered in a powder collection vessel. Resulting particles have a VMGD and an FPF within expected ranges. Powder is filled at approximately 8.0-mg quantities into size 2 hydroxypropylmethyl cellulose (HPMC) capsules and then will be packaged in Aclar-foil blister cards. The blister cards are sealed in aluminum foil bags, containing a small, food-grade desiccant bag for additional moisture protection.

While this invention has been particularly shown and described with references to preferred embodiments thereof, it will be understood by those skilled in the art that various changes in form and details may be made therein without departing from the scope of the invention encompassed by the appended claims.

### EXAMPLE 2

Using the process (modified by adding leucine for leucine-containing formulations) described in Example 1, the following formulations are made:

| | DPPC | PTH | Citrate | Leucine |
|---|---|---|---|---|
| LEUCINE FREE | 60-94 | 1-20 | 5-20 | — |
| | 85 | 5 | 10 | — |
| | 80 | 10 | 10 | — |
| | 70 | 20 | 10 | — |
| LEUCINE | 1-89 | 1-20 | 5-20 | 5-90 |
| CONTAINING | | | | |
| | 16 | 12.5 | 10 | 55.5 |
| | 13 | 6 | 10 | 71 |
| | 12 | 16 | 10 | 62 |

1. A formulation having particles comprising, by weight, from about 60 to about 94% DPPC; from about 1 to about 20% PTH; and from about 5 to about 20% sodium citrate.

2. A formulation having particles comprising, by weight, about 85% DPPC; about 5% PTH; and about 10% sodium citrate.

3. A formulation having particles comprising, by weight, about 80% DPPC; about 10% PTH; and about 10% sodium citrate.

4. A formulation having particles comprising, by weight, about 70% DPPC; about 20% PTH; and about 10% sodium citrate.

5. A formulation having particles comprising, by weight, from about 1 to about 89% DPPC; from about 1 to about 20% PTH; from about 5 to about 20% sodium citrate; and from about 5 to about 90% leucine.

6. A formulation having particles comprising, by weight, about 16% DPPC; about 12.5% PTH; about 10% sodium citrate; and about 55.5% leucine.

7. A formulation having particles comprising, by weight, about 13% DPPC; about 6% PTH; about 10% sodium citrate; and about 71% leucine.

8. A formulation having particles comprising, by weight, about 12% DPPC; about 16% PTH; about 10% sodium citrate; and about 62% leucine.

9. The formulation of claim 1 or 5, wherein the particles comprise a mass of from about 1.5 mg to about 20 mg per receptacle.

10. The formulation of claim 9, wherein the particles comprise a mass of about 50 micrograms to about 2 mg of PTH per receptacle.

11. The formulation of claim 9, wherein the particles comprise a mass of about 100 micrograms to about 1 mg of PTH per receptacle.

12. The formulation of claim 9, wherein the particles have a tap density less than about 0.4 g/cm³.

13. The formulation of claim 9, wherein the particles have a tap density less than about 0.1 g/cm³.

14. The formulation of claim 9, wherein the particles have a median geometric diameter of from about 5 micrometers to about 30 micrometers.

15. The formulation of claim 14, wherein the particles have a median geometric diameter of from about 6 micrometers to about 8 micrometers.

16. The formulation of claim 9, wherein the particles have an aerodynamic diameter of from about 1 micrometer to about 5 micrometers.

17. The formulation of claim 16, wherein the particles have an aerodynamic diameter of from about 1 micrometer to about 3 micrometers.

18. The formulation of claim 16, wherein the particles have an aerodynamic diameter of from about 3 micrometers to about 5 micrometers.

19. A method for treating a human patient in need of parathyroid hormone comprising administering pulmonarily to the respiratory tract of a patient in need of treatment, an effective amount of particles comprising by weight from about 60 to about 94% DPPC; from about 1 to about 20% PTH; and from about 5 to about 20% sodium citrate wherein release of the PTH is rapid.

20. A method for treating a human patient in need of parathyroid hormone comprising administering pulmonarily to the respiratory tract of a patient in 10 need of treatment, an effective amount of particles comprising by weight, from about 1 to about 89% DPPC; from about 1 to about 20% PTH; from about 5 to about 10% sodium citrate; and from about 5 to about 90% leucine wherein release of the PTH is rapid.

21. A method of delivering an effective amount of PTH to the pulmonary system, comprising:
a) providing a mass of particles comprising by weight, from about 60 to about 94% DPPC; from about 1 to about 20% PTH; and from about 5 to about 20% sodium citrate; and
b) administering via simultaneous dispersion and inhalation the particles, from a receptacle having the mass of the particles, to a human subject's respiratory tract, wherein release of the PTH is rapid.

22. A method of delivering an effective amount of PTH to the pulmonary system, comprising:
a) providing a mass of particles comprising by weight, from about 1 to about 89% DPPC; from about 1 to about 20% PTH; from about 5 to about 20% sodium citrate; and from about 5 to about 90% leucine; and
b) administering via simultaneous dispersion and inhalation the particles, from a receptacle having the mass of the particles, to a human subject's respiratory tract, wherein release of the PTH is rapid.

23. The formulation of claim 1 or 5, wherein the particles further comprise a low transition temperature phospholipid.

24. A kit for administration of PTH comprising two or more receptacles, wherein said receptacles comprise unit dosages of particles comprising, by weight, , from about 60 to about 94% DPPC; from about 1 to about 20% PTH; and from about 5 to about 20% sodium citrate.

25. The kit of claim 58, wherein two or more receptacles comprise unit dosages of particles comprising, by weight, from about 1 to about 89% DPPC; from about 1 to about 20% PTH; from about 5 to about 20% sodium citrate and from about 5 to about 90% leucine and wherein one or more receptacles comprise unit dosages of particles comprising, by weight, 75% to 80% DPPC, 10% to 15% PTH and 10% sodium citrate.

26. The kit of claim 24 or 25, wherein the unit dosages of said two or more receptacles is the same.

27. The kit of claim 24 or 25, wherein the unit dosages of said two or more receptacles is different.

28. The kit of claim 24 or 25, wherein said kit further comprises instructions for use of said two or more receptacles.

29. A pharmaceutical composition comprising the formulation of claim 1 or 5.

30. The pharmaceutical composition of claim 29 further comprising an antiresorptive agent.

31. The antiresorptive agent of claim 30 selected from the group consisting of bisphosphonates, estrogens; selective estrogen receptor modulators (SERM), raloxifene, and calcitonin.

32. The antiresorptive agent of claim 31, wherein the bisphosphonate is selected from the group consisting of alendronate and risedronate.

33. The formulation of claim 17, wherein the particles are not aggregated as they exit the inhaler.

34. The formulation of claim 18, wherein the particles are not aggregated as they exit the inhaler.
